(19) United States
(12) Patent Application Publication
(10) Pub. No.: US 2016/0256654 A1
(43) Pub. Date: Sep. 8, 2016

(54) Expandable Tubing System for Delivering Gases

(71) Applicants: Frances Elaine Dixon, (US); Gregory Lawrence Foley, (US)

(72) Inventors: John Brian McAdams, Lauderdale By The Sea, FL (US); Amos Spalter, Miami, FL (US)

(21) Appl. No.: 15/060,569

(22) Filed: Mar. 3, 2016

Related U.S. Application Data
(60) Provisional application No. 62/127,428, filed on Mar. 3, 2015.

(51) Int. Cl. A61M 16/06; A61M 16/12; A61M 16/20; A61M 16/00

(52) U.S. Cl. CPC A61M 16/0666; A61M 16/0003; A61M 16/12; A61M 16/20; A61M 16/0688; A61M 2016/003; A61M 2205/3334

(57) ABSTRACT

An expandable tubing system for delivering gases delivers various gases to patients. The system includes at least one expandable tube and a nasal cannula. The expandable tube includes a main tube which delivers gases from a gas supply to the nasal cannula or another device. The at least one expandable tube each include a first coupler, a second coupler, an inner tube, and an outer cover. The first coupler and the second coupler allow the inner tube to be connected in between two devices. The inner tube expands when filled with gases and retracts when emptied. The outer cover encircles and protects the inner tube. The nasal cannula includes a cannula coupler, a cannula tube, and a nosepiece. The cannula coupler connects the nasal cannula to the main tube. The cannula tube is transports gases from the cannula coupler to the nosepiece, which directs gases into the patient's nose.

The current application claims a priority to the U.S. Provisional Patent application Ser. No. 62/127,428 filed on Mar. 3, 2015.

### FIELD OF THE INVENTION

The present invention generally relates to devices that are used to deliver oxygen or other gases to people who experience difficulties breathing, or patients of medical procedures. More specifically, the present invention relates to a delivery system that incorporates expandable tubing and a nasal cannula that adheres to the user's nose and improves airflow. When not in use, the tubing retracts to minimize clutter and prevent tangling.

### BACKGROUND OF THE INVENTION

Many people, including elderly people and those who suffer from respiratory disabilities or complications, require a supplemental supply of oxygen. Those undergoing surgery or similar medical procedures also require a supply of anesthetic. Current systems use great lengths of tubing to deliver gases from an oxygen tank or oxygen concentrator to the patient's mouth or nose. Because of this, it is not uncommon for tubes to become tangled. In addition, current nasal cannula devices are able to deliver oxygen to a person's nose; however, they do not optimize the amount of oxygen that flows into the nose.

Accordingly, there is a present need for a system which may be used to supply a person with oxygen or other gases, while minimizing the risk of tangling tubes. Moreover, there is a present need for said system to optimize the amount of oxygen that flows into the nose. The present invention makes use of tubes which are able to expand to a certain length during use, and retract to a relatively short and manageable length when not in use. The present invention also includes a nasal cannula which uses an adhesive strip that expands airways and improves airflow.

### BRIEF DESCRIPTION OF THE DRAWINGS

FIG. 1 is a front view of the present invention.

FIG. 2 is a front view of the expandable tube in a retracted configuration.

FIG. 3 is a front schematic view of the expandable tube in the retracted configuration.

FIG. 4 is a front view of the expandable tube in an extended configuration.

FIG. 5 is a front schematic view of the expandable tube in the extended configuration.

FIG. 6 is a front view of the nasal cannula.

FIG. 7 is a front view of the preferred embodiment of the nosepiece, wherein the preferred embodiment of the nosepiece comprises a nasal strip.

FIG. 8 is a rear view of the preferred embodiment of the nosepiece.

FIG. 9 is a front view of an alternative embodiment of the nosepiece, wherein the alternative embodiment of the nosepiece comprises a head strap.

FIG. 10 is a front view of the valve.

### DETAILED DESCRIPTION OF THE INVENTION

All illustrations of the drawings are for the purpose of describing selected versions of the present invention and are not intended to limit the scope of the present invention.

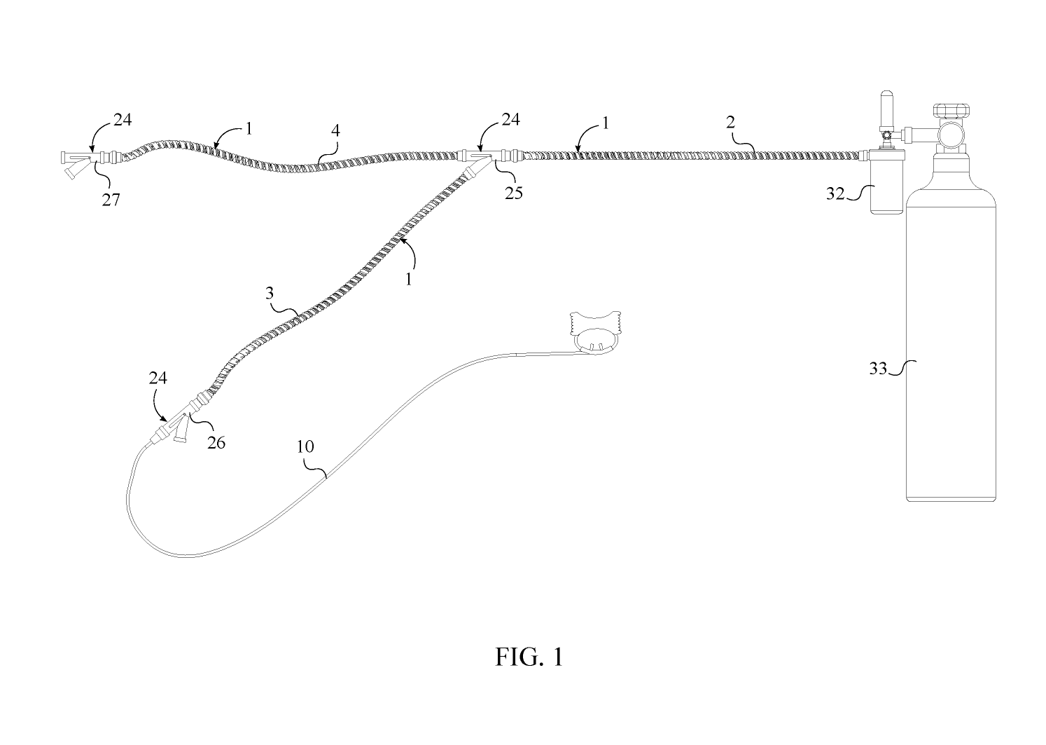

With reference to FIGS. 1-3, the present invention is an expandable tubing system for delivering gases. Specifically, the present invention may be used to deliver oxygen, medical air, nitrous oxide, and other gases for the purposes of aiding breathing, anesthesia, or sedation. The present invention comprises at least one expandable tube 1 and a nasal cannula 10. The at least one expandable tube 1 is used to transfer gases to a patient. The expandable tube 1 is designed to expand when filled with gas and retract when not in use. This helps to limit the possibility of the expandable tube 1 getting tangled. In the preferred embodiment of the present invention, the at least one expandable tube 1 comprises a main tube 2 which can be used to direct gases to the nasal cannula 10 or another device. Each of the at least one expandable tube 1 comprises an inner tube 5, an outer cover 6, a first coupler 7, and a second coupler 8. The first coupler 7 and the second coupler 8 are used to make connections between the expandable tube 1 and a gas supply 33, a valve, or another device. The first coupler 7 and the second coupler 8 are in fluid communication with each other through the inner tube 5. The first coupler 7 and the second coupler 8 are positioned opposite to each other along the inner tube 5.

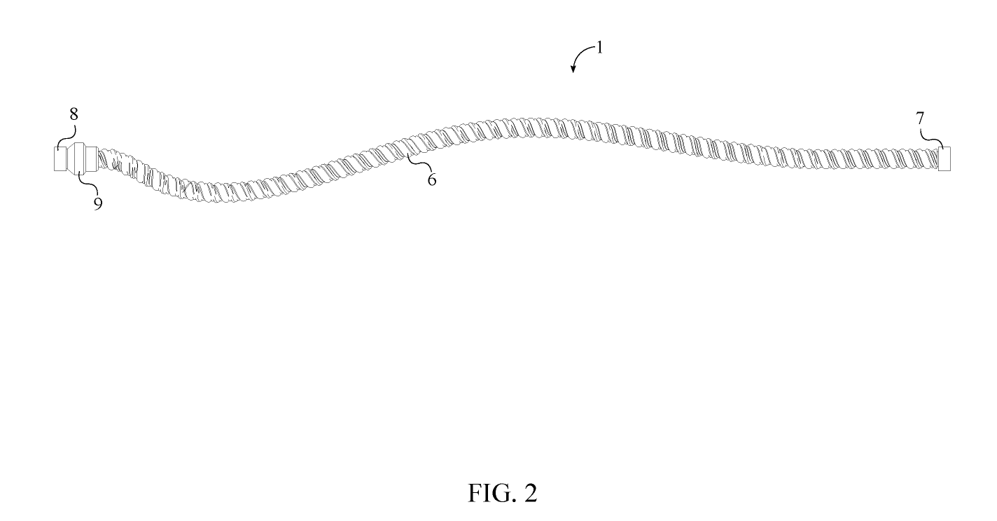

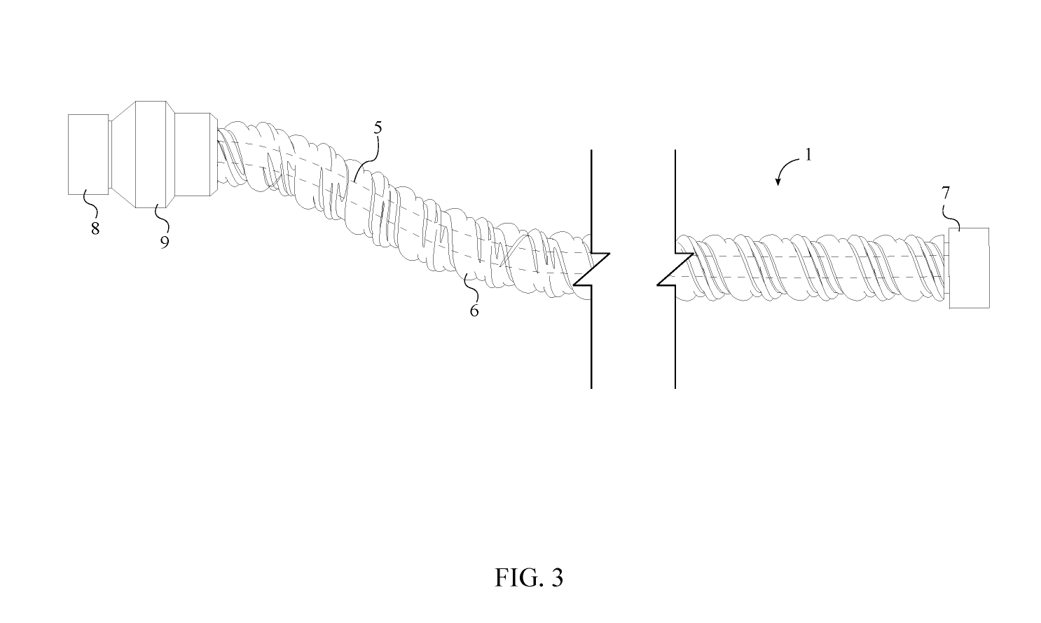

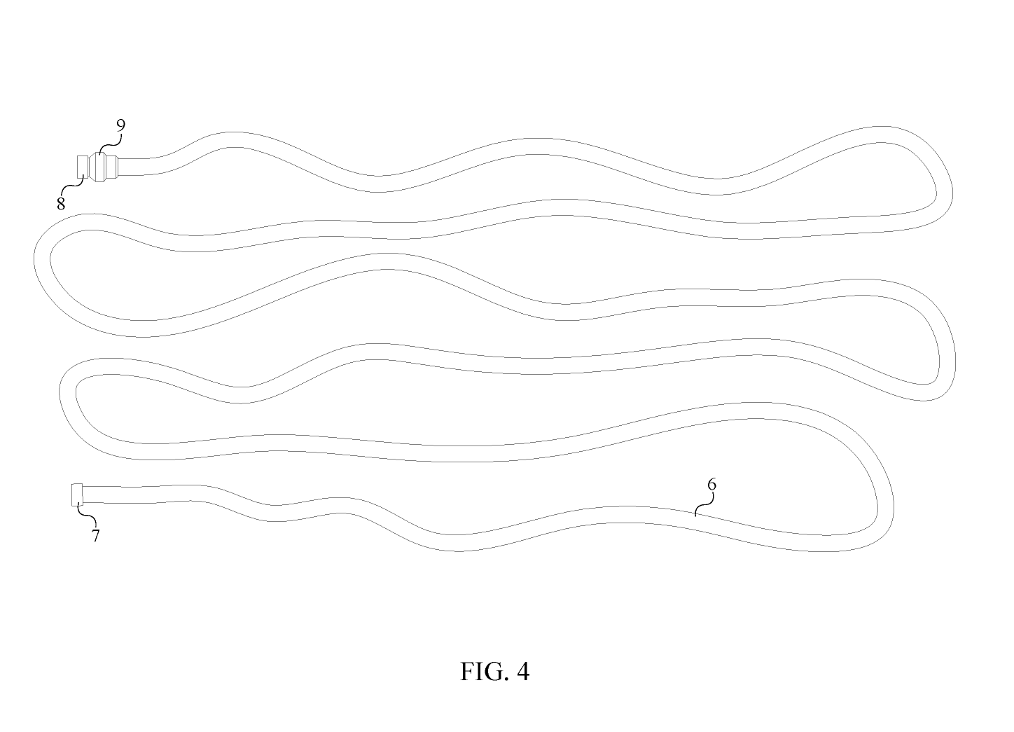

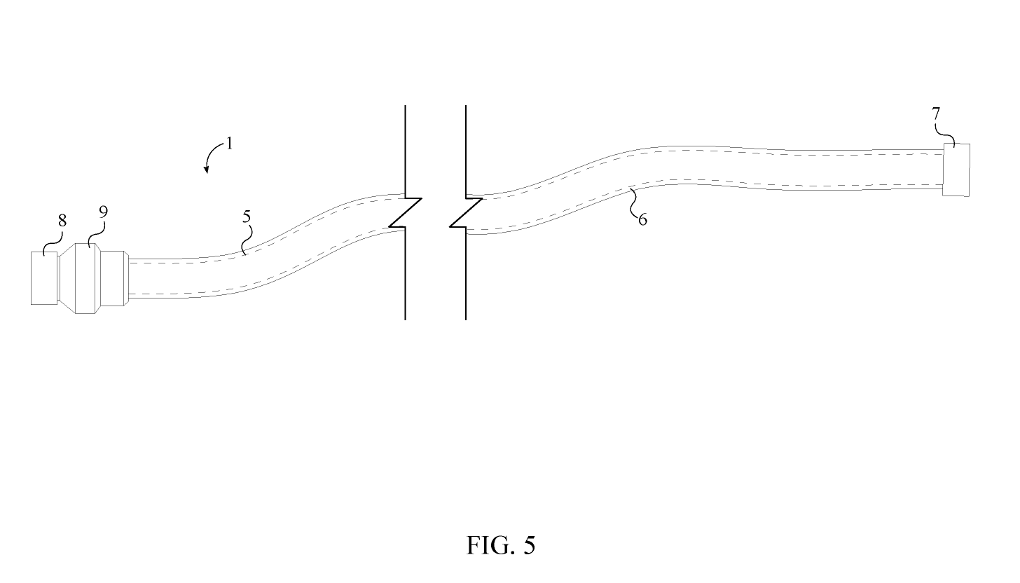

In reference to FIG. 3, the inner tube 5 is the conduit through which oxygen or other gases are able to flow. When filled with gases, the inner tube 5 expands in length and diameter until the expandable tube 1 is in an expanded configuration. This is shown in FIGS. 4-5. When not in use, gases are permitted to escape the inner tube 5 so that the expandable tube 1 returns to a retracted configuration. This is shown in FIGS. 2-3. In the preferred embodiment, the inner tube 5 is made from rubber; however, any other suitable material may be used in alternative embodiments. The outer cover 6 encircles the inner tube 5 from the first coupler 7 to the second coupler 8. The outer cover 6 is used as a protective barrier for the inner tube 5, preventing the inner tube 5 from being punctured or otherwise damaged. The protective barrier also helps to prevent the inner tube 5 from expanding too far as a result of excess pressure within the inner tube 5. To allow the inner tube 5 to retract, the outer cover 6 is able to fold and wrinkle such that both components have the same length. In the preferred embodiment, the outer cover 6 is made from nylon; however, any other suitable material may alternatively be used. Further, in the preferred embodiment of the present invention, the outer cover 6 is color coded to match a specific length. For example, expandable tubes which expand to 10, 25, and 50 feet may incorporate outer covers with the colors blue, green, and orange, respectively. Alternatively, any other color and length combinations may be used.

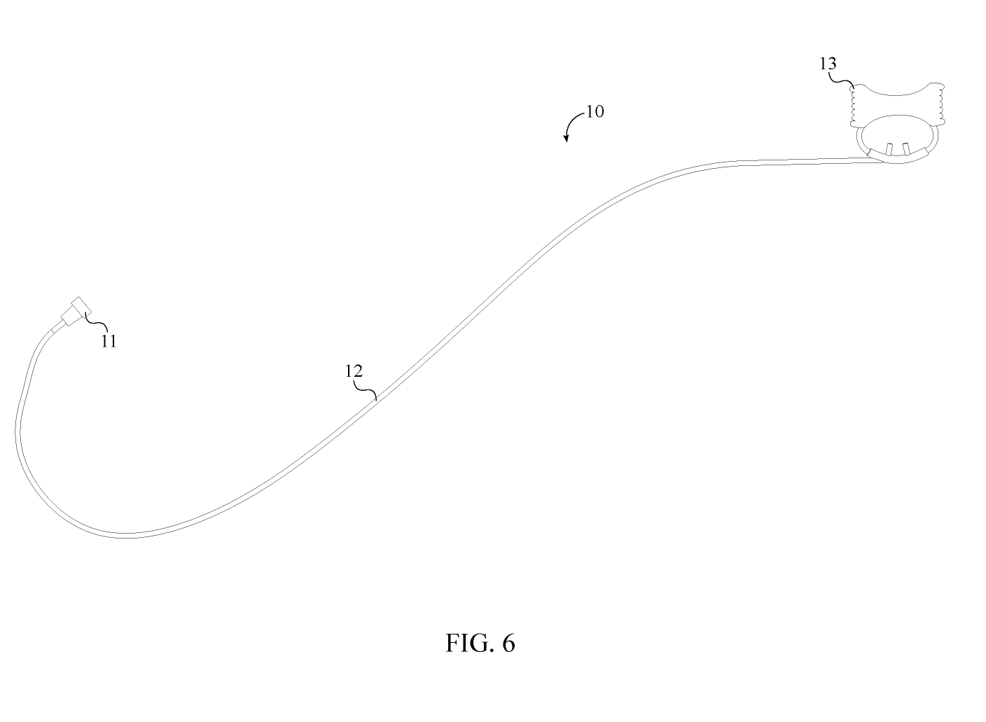

In reference to FIG. 6, the nasal cannula 10 comprises a cannula coupler 11, a cannula tube 12, and a nosepiece 13. The nasal cannula 10 is used to direct oxygen or other gases into the nose of the patient. The cannula coupler 11 and the nosepiece 13 are in fluid communication with each other through the cannula tube 12. The cannula coupler 11 is used to form a hermetic connection between the nasal cannula 10 tube and a valve or the expandable tube 1. The cannula tube 12 is used to deliver gases from the cannula coupler 11 to the nosepiece 13. The cannula coupler 11 and the nosepiece 13 are positioned opposite to each other along the cannula tube 12. The nosepiece 13 is used to direct gases into the nose of the patient and may be mounted to the face of the patient. The cannula coupler 11 is in fluid communication with the second coupler 8 of the main tube 2, allowing gases to flow from the main tube 2, through the cannula tube 12, out the nosepiece 13 and into the nose of the patient.

In the preferred embodiment of the present invention, each of the at least one expandable tube 1 comprises a pressure-regulating valve 9. In reference to FIG. 2, the pressure-regulating valve 9 is used to maintain a constant pressure throughout the present invention. The first coupler 7 and the second coupler 8 are in fluid communication with each other through the inner tube 5 and the pressure-regulating valve 9. In the preferred embodiment, the pressure-regulating valve 9 is positioned adjacent to the second coupler 8; however, in alternative embodiments, the pressure-regulating valve 9 may be incorporated at any location on the expandable tube 1. Because the pressure inside the expandable tube 1 is what allows the inner tube 5 to increase in size, if the pressure falls too low, the expandable tube 1 can begin to retract. Moreover, if the pressure is too high, the inner tube 5 can expand too far or rupture. In the preferred embodiment, the pressure-regulating valve 9 automatically adjusts the pressure throughout the expandable tube 1; however, in alternative embodiments, this may be done manually.

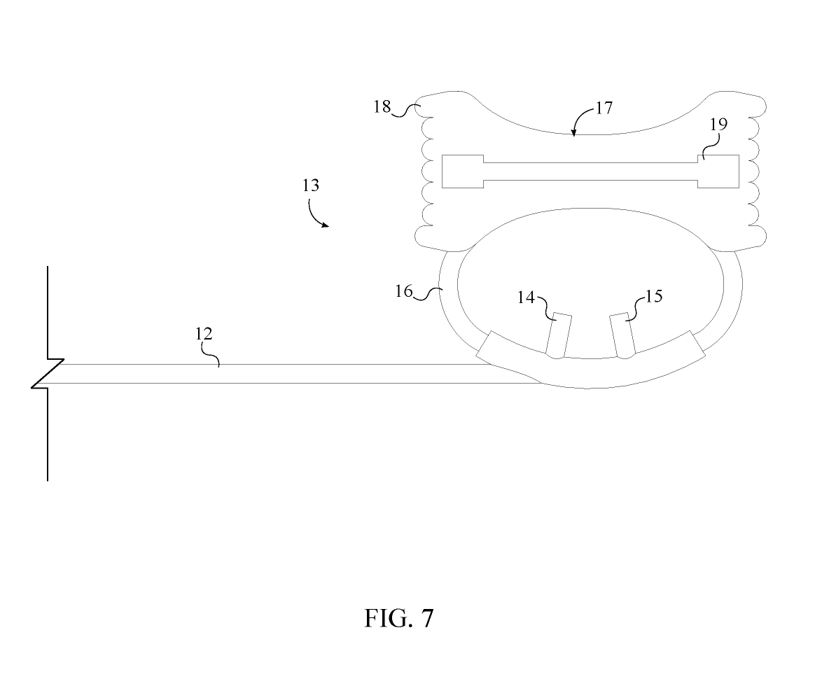

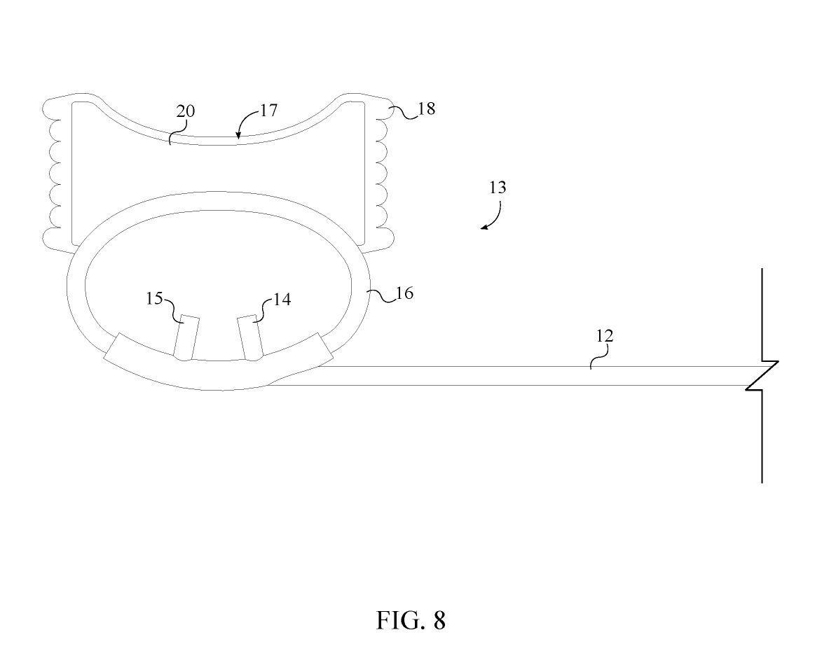

In reference to FIGS. 7-8, the nosepiece 13 comprises a first nasal insert 14, a second nasal insert 15, a circulation ring 16, and a nasal strip 17. The cannula tube 12 is in fluid communication with the circulation ring 16. The circulation ring 16 is used with the nasal strip 17 to properly position the first nasal insert 14 and the second nasal insert 15 on the face of the patient. The first nasal insert 14 and the second nasal insert 15 are in fluid communication with the circulation ring 16 so that the circulation ring 16 may equally distribute gases to the first nasal insert 14 and the second nasal insert 15. The cannula tube 12 is positioned outside the circulation ring 16 and delivers gases to the circulation ring 16. The first nasal insert 14 and the second nasal insert 15 are positioned inside the circulation ring 16 and are angularly offset from each other about the circulation ring 16. This configuration allows gases to be directed into the nose of the patient. The nasal strip 17 is perimetrically connected about the circulation ring 16 and is used to mount the nosepiece 13 to the patient's face. The nasal strip 17 is also used to expand the airways of the patient's nose so that the patient may breathe easier. The nasal strip 17 and the cannula tube 12 are diametrically opposed to each other about the circulation ring 16. This configuration is designed such that the nasal strip 17 may be mounted over the bridge of the patient's nose, while the first nasal insert 14 and the second nasal insert 15 are placed adjacent to or within the patient's nostrils.

In reference to FIG. 7-8, the nasal strip 17 comprises a nose pad 18, at least one flexible rib 19, and an adhesion layer 20. The nose pad 18 provides a comfortable means of attaching the nasal strip 17 to the nose of the patient. In the preferred embodiment of the nasal strip 17, the nose pad 18 is made from a cloth-like material. The at least one flexible rib 19 is integrated through the nose pad 18 and is used to open the patient's airways. The elasticity of the flexible rib 19 allows the nasal strip 17 to pull on the patient's nose after the nasal strip 17 is mounted onto the bridge of the patient's nose. The adhesion layer 20 is superimposed across the nasal strip 17 and allows the nasal strip 17 to adhere to the patient's nose and pull open the patient's airways.

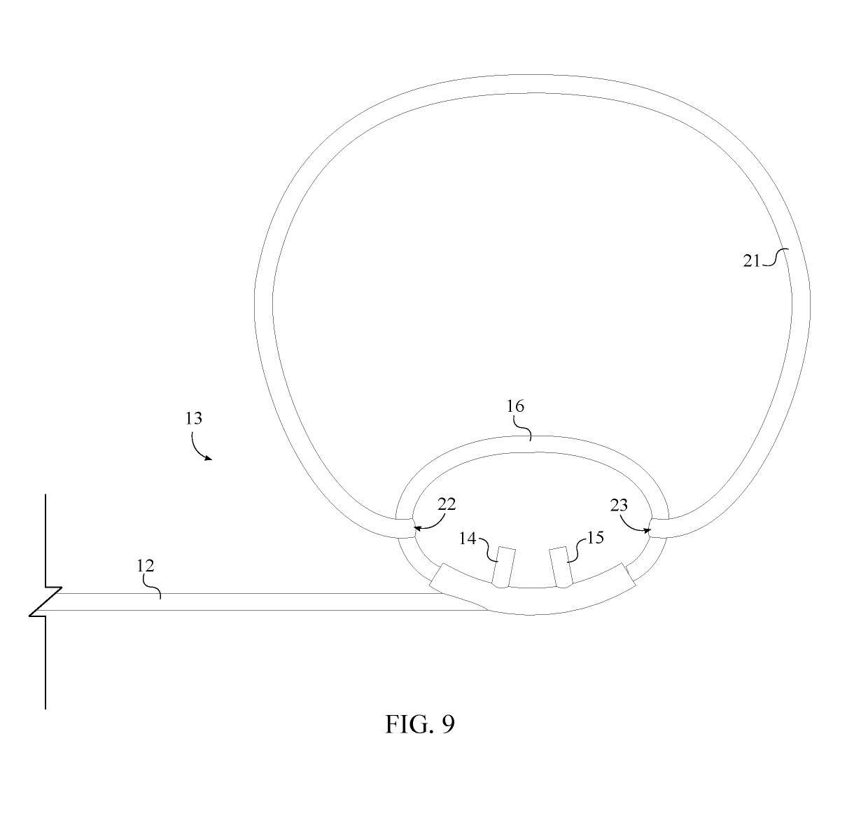

In an alternative embodiment of the present invention, shown in FIG. 9, the nosepiece 13 comprises a head strap 21 instead of the nasal strip 17. The head strap 21 may be positioned around the patient's head or neck to secure the nasal cannula 10 to the user. The head strap 21 comprises a first end 22 and a second end 23. The first end 22 and the second end 23 are both connected outside the circulation ring 16. The first end 22 and the second end 23 are angularly offset from each other about the circulation ring 16 so that the head strap 21 may be easily positioned about the patient's head or neck.

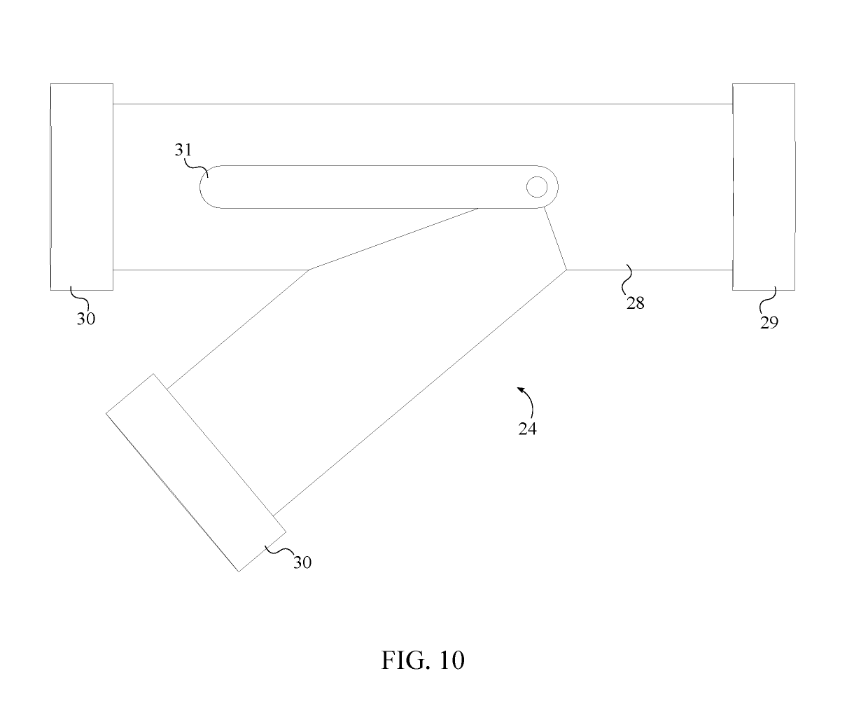

In reference to FIG. 1 and FIG. 10, the present invention further comprises at least one valve 24. The at least one valve 24 is used to control the flow of gases through the present invention. Further, the at least one valve 24 may be used to toggle between delivering gases to the nasal cannula 10 and another device. Each of the at least one valve 24 comprises a valve body 28, an inlet 29, a plurality of outlets 30, and a directional control knob 31. The inlet 29 is in fluid communication with each of the plurality of outlets 30 through the valve body 28. In the preferred embodiment of the at least one valve 24, there are two outlets 30; however, any number of outlets 30 may be used, based on the needs of the patient. The directional control knob 31 is operatively integrated into the valve body 28 and is used to direct fluid flow from the inlet 29 to a selected outlet from the plurality of outlets 30.

In reference to FIG. 1 and FIG. 10, the at least one valve 24 comprises a main valve 25. The second coupler 8 of the main tube 2 is in fluid communication with the inlet 29 of the main valve 25. The cannula coupler 11 is in fluid communication with the selected outlet of the main valve 25. This configuration allows the main valve 25 to be used to direct gases from the main tube 2, to the nasal cannula 10, or to another device.

In reference to FIG. 1 and FIG. 10, the at least one expandable tube 1 further comprises a first auxiliary tube 3 and the at least one valve 24 comprises a first auxiliary valve 26. The first auxiliary tube 3 allows nasal cannula 10 to be used further away from a gas supply 33. Also, the first auxiliary tube 3 allows the nasal cannula 10 to be distanced from the main valve 25. The first coupler 7 of the first auxiliary tube 3 is in fluid communication with the selected outlet 30 of the main valve 25. The first auxiliary valve 26 is used to provide added control over the flow of gases through the present invention. For example, the main valve 25 may be used to select the nasal cannula 10 or another device, while the first auxiliary valve 26 may be used to adjust the flow of gases to the nasal cannula 10. Accordingly, the second coupler 8 of the first auxiliary tube 3 is in fluid communication with the inlet 29 of the first auxiliary valve 26 and the cannula coupler 11 is in fluid communication with the selected outlet 30 of the first auxiliary valve 26.

In reference to FIG. 1 and FIG. 10, the at least one expandable tube 1 further comprises a second auxiliary tube 4 and the at least one valve 24 comprises a second auxiliary valve 27. The second auxiliary tube 4 and the second auxiliary valve 27 are used to connect a second device to the present invention. The first coupler 7 of the second auxiliary tube 4 is in fluid communication with another outlet of the main valve 25, wherein the other outlet 30 is one of the plurality of outlets 30. The second coupler 8 of the second auxiliary tube 4 is in fluid communication with the inlet 29 of the second auxiliary valve 27. This configuration allows the main valve 25 to be used to select the nasal cannula 10 or a second device, while the second auxiliary valve 27 may be used to adjust the flow of gases to the second device.

In reference to FIG. 1 and FIG. 10, the present invention further comprises a flow meter 32 and a gas supply 33. The gas supply 33 is used to pump oxygen, medical air, nitrous oxide, or other gases through the expandable tube 1 and to the patient. In the preferred embodiment of the present invention, the gas supply 33 is a gas canister; however, an air compressor or any other source may be used. The flow meter 32 is used to monitor the flowrate of gases being pumped through the present invention. The first coupler 7 of the main tube 2 is in fluid communication with the flow meter 32 and the gas supply 33 is in fluid communication with the first coupler 7 of the main tube 2 through the flow meter 32. While this configuration represents the preferred embodiment of the present invention, the present invention may also work without the use of the flow meter 32. In this situation, the gas supply 33 is in direct fluid communication with the first coupler 7 of the main tube 2.

Although the invention has been explained in relation to its preferred embodiment, it is to be understood that many other possible modifications and variations can be made without departing from the spirit and scope of the invention as hereinafter claimed.

What is claimed is:

1. An expandable tubing system for delivering gases comprises:
at least one expandable tube;
a nasal cannula;
the at least one expandable tube comprises a main tube;
each of the at least one expandable tube comprises an inner tube, an outer cover, a first coupler and a second coupler;
the nasal cannula comprises a cannula coupler, a cannula tube, and a nosepiece;
the first coupler and the second coupler being in fluid communication with each other through the inner tube;
the first coupler and the second coupler being positioned opposite to each other along the inner tube;
the outer cover encircling the inner tube from the first coupler to the second coupler;
the cannula coupler and the nosepiece being in fluid communication with each other through the cannula tube;
the cannula coupler and the nosepiece being positioned opposite to each other along the cannula tube; and
the cannula coupler being in fluid communication with the second coupler of the main tube.

2. The expandable tubing system for delivering gases as claimed in claim 1 comprises:
each of the at least one expandable tube further comprises a pressure-regulating valve; and
the first coupler and the second coupler being in fluid communication with each other through the inner tube and the pressure-regulating valve.

3. The expandable tubing system for delivering gases as claimed in claim 1 comprises:
the nosepiece comprises a first nasal insert, a second nasal insert, a circulation ring, and a nasal strip;
the cannula tube being in fluid communication with the circulation ring;
the first nasal insert and the second nasal insert being in fluid communication with the circulation ring;
the cannula tube being positioned outside the circulation ring;
the first nasal insert and the second nasal insert being positioned inside the circulation ring;
the first nasal insert and the second nasal insert being angularly offset from each other about the circulation ring;
the nasal strip being perimetrically connected about the circulation ring; and
the nasal strip and the cannula tube being diametrically opposed to each other about the circulation ring.

4. The expandable tubing system for delivering gases as claimed in claim 3 comprises:
the nasal strip comprises a nose pad, at least one flexible rib, and an adhesion layer;
the at least one flexible rib being integrated through the nose pad; and
the adhesion layer being superimposed across the nasal strip.

5. The expandable tubing system for delivering gases as claimed in claim 1 comprises:
the nosepiece comprises a first nasal insert, a second nasal insert, a circulation ring, and a head strap;
the head strap comprises a first end and a second end;
the cannula tube being in fluid communication with the circulation ring;
the first nasal insert and the second nasal insert being in fluid communication with the circulation ring;
the cannula tube being positioned outside the circulation ring;
the first nasal insert and the second nasal insert being positioned inside the circulation ring;
the first nasal insert and the second nasal insert being angularly offset from each other about the circulation ring;
the first end being connected outside the circulation ring;
the second end being connected outside the circulation ring; and
the first end and the second end being angularly offset from each other about the circulation ring.

6. The expandable tubing system for delivering gases as claimed in claim 1 comprises:
at least one valve;
each of the at least one valve comprises a valve body, an inlet, a plurality of outlets, and a directional control knob;
the inlet being in fluid communication with each of the plurality of outlets through the valve body; and
the directional control knob being operatively integrated into the valve body, wherein the directional control knob is used to direct fluid flow from the inlet to a selected outlet from the plurality of outlets.

7. The expandable tubing system for delivering gases as claimed in claim 6 comprises:
the at least one valve comprises a main valve;
the second coupler of the main tube being in fluid communication with the inlet of the main valve; and
the cannula coupler being in fluid communication with the selected outlet of the main valve.

8. The expandable tubing system for delivering gases as claimed in claim 6 comprises:
the at least one expandable tube further comprises a first auxiliary tube;
the at least one valve comprises a main valve and a first auxiliary valve;
the second coupler of the main tube being in fluid communication with the inlet of the main valve;
the first coupler of the first auxiliary tube being in fluid communication with the selected outlet of the main valve;
the second coupler of the first auxiliary tube being in fluid communication with the inlet of the first auxiliary valve; and
the cannula coupler being in fluid communication with the selected outlet of the first auxiliary valve.

9. The expandable tubing system for delivering gases as claimed in claim 6 comprises:
the at least one expandable tube further comprises a second auxiliary tube;
the at least one valve comprises a main valve and a second auxiliary valve;
the second coupler of the main tube being in fluid communication with the inlet of the main valve;
the first coupler of the second auxiliary tube being in fluid communication with another outlet of the main valve, wherein the other outlet is one of the plurality of outlets; and
the second coupler of the second auxiliary tube being in fluid communication with the inlet of the second auxiliary valve.

10. The expandable tubing system for delivering gases as claimed in claim 1 comprises:
a flow meter; and
the first coupler of the main tube being in fluid communication with the flow meter.

11. The expandable tubing system for delivering gases as claimed in claim 10 comprises:
a gas supply; and
the gas supply being in fluid communication with the first coupler of the main tube through the flow meter.

12. The expandable tubing system for delivering gases as claimed in claim 1 comprises:
a gas supply; and
the gas supply being in fluid communication with the first coupler of the main tube.

13. An expandable tubing system for delivering gases comprises:
at least one expandable tube;
a nasal cannula;
the at least one expandable tube comprises a main tube;
each of the at least one expandable tube comprises an inner tube, an outer cover, a first coupler and a second coupler;
the nasal cannula comprises a cannula coupler, a cannula tube, and a nosepiece;
the nosepiece comprises a first nasal insert, a second nasal insert, a circulation ring, and a nasal strip;
the first coupler and the second coupler being in fluid communication with each other through the inner tube;
the first coupler and the second coupler being positioned opposite to each other along the inner tube;
the outer cover encircling the inner tube from the first coupler to the second coupler;
the cannula coupler and the nosepiece being in fluid communication with each other through the cannula tube;
the cannula coupler and the nosepiece being positioned opposite to each other along the cannula tube;
the cannula coupler being in fluid communication with the second coupler of the main tube;
the cannula tube being in fluid communication with the circulation ring;
the first nasal insert and the second nasal insert being in fluid communication with the circulation ring;
the cannula tube being positioned outside the circulation ring;
the first nasal insert and the second nasal insert being positioned inside the circulation ring;
the first nasal insert and the second nasal insert being angularly offset from each other about the circulation ring;
the nasal strip being perimetrically connected about the circulation ring; and
the nasal strip and the cannula tube being diametrically opposed to each other about the circulation ring.

14. The expandable tubing system for delivering gases as claimed in claim 13 comprises:
each of the at least one expandable tube further comprises a pressure-regulating valve; and
the first coupler and the second coupler being in fluid communication with each other through the inner tube and the pressure-regulating valve.

15. The expandable tubing system for delivering gases as claimed in claim 13 comprises:
the nasal strip comprises a nose pad, at least one flexible rib, and an adhesion layer;
the at least one flexible rib being integrated through the nose pad; and
the adhesion layer being superimposed across the nasal strip.

16. The expandable tubing system for delivering gases as claimed in claim 13 comprises:
the nosepiece comprises a first nasal insert, a second nasal insert, a circulation ring, and a head strap;
the head strap comprises a first end and a second end;
the cannula tube being in fluid communication with the circulation ring;
the first nasal insert and the second nasal insert being in fluid communication with the circulation ring;
the cannula tube being positioned outside the circulation ring;
the first nasal insert and the second nasal insert being positioned inside the circulation ring;
the first nasal insert and the second nasal insert being angularly offset from each other about the circulation ring;
the first end being connected outside the circulation ring;
the second end being connected outside the circulation ring; and
the first end and the second end being angularly offset from each other about the circulation ring.

17. The expandable tubing system for delivering gases as claimed in claim 1 comprises:
at least one valve;
the at least one valve comprises a main valve;
each of the at least one valve comprises a valve body, an inlet, a plurality of outlets, and a directional control knob;
the inlet being in fluid communication with each of the plurality of outlets through the valve body;
the directional control knob being operatively integrated into the valve body, wherein the directional control knob is used to direct fluid flow from the inlet to a selected outlet from the plurality of outlets;
the second coupler of the main tube being in fluid communication with the inlet of the main valve; and
the cannula coupler being in fluid communication with the selected outlet of the main valve.

18. The expandable tubing system for delivering gases as claimed in claim 6 comprises:
the at least one expandable tube further comprises a first auxiliary tube;
the at least one valve further comprises a first auxiliary valve;
the second coupler of the main tube being in fluid communication with the inlet of the main valve;
the first coupler of the first auxiliary tube being in fluid communication with the selected outlet of the main valve;
the second coupler of the first auxiliary tube being in fluid communication with the inlet of the first auxiliary valve; and
the cannula coupler being in fluid communication with the selected outlet of the first auxiliary valve.

19. The expandable tubing system for delivering gases as claimed in claim 6 comprises:
the at least one expandable tube further comprises a second auxiliary tube;
the at least one valve further comprises a second auxiliary valve;
the second coupler of the main tube being in fluid communication with the inlet of the main valve;
the first coupler of the second auxiliary tube being in fluid communication with another outlet of the main valve, wherein the other outlet is one of the plurality of outlets; and
the second coupler of the second auxiliary tube being in fluid communication with the inlet of the second auxiliary valve.

20. The expandable tubing system for delivering gases as claimed in claim 1 comprises:
a flow meter;
a gas supply;
the first coupler of the main tube being in fluid communication with the flow meter; and
the gas supply being in fluid communication with the first coupler of the main tube through the flow meter.
